(19) United States
(12) Patent Application Publication
(10) Pub. No.: US 2008/0189047 A1
(43) Pub. Date: Aug. 7, 2008

(54) METHOD AND SYSTEM FOR GENETIC RESEARCH USING GENETIC SAMPLING VIA AN INTERACTIVE ONLINE NETWORK

(75) Inventors: June T. W. Wong, Vancouver (CA); Clement K.L. Ma, Vancouver (CA)

Correspondence Address: OYEN, WIGGS, GREEN & MUTALA LLP;480 - THE STATION, 601 WEST CORDOVA STREET, VANCOUVER, BC, V6B 1G1, omitted

(73) Assignee: 0752004 B.C. LTD., Vancouver (CA)

(21) Appl. No.: 11/934,024

(22) Filed: Nov. 1, 2007

Related U.S. Application Data
(60) Provisional application No. 60/863,956, filed on Nov. 1, 2006.

(51) Int. Cl. G01N 33/48

(52) U.S. Cl. 702/20

(57) ABSTRACT

The invention provides a method and system for conducting genetic research using DNA sampling, preferably via an online social network, using a set of family members related by defined genealogical relationships to identify carriers of a DNA of interest for obtaining DNA information. According to various aspects of the invention, the method can be used for identifying and determining carriers of a DNA of interest, and further genealogical analysis.

### REFERENCE TO RELATED APPLICATION

This application claims priority from U.S. provisional patent application Ser. No. 60/863,956 filed Nov. 1, 2006, which is pending.

### TECHNICAL FIELD

The invention relates to the field of genetic research and genealogy and more particularly to a method of identifying carriers of specific genes and DNA markers.

### BACKGROUND

The use of DNA analysis for genetic research, such as for diagnosing disease, alerting individuals who have a likelihood of carrying a genetically-transmitted disease, or for forensic or genealogical purposes, has become more prevalent in recent years due to improvements in techniques for such analysis. Genetic researchers will often be posed with the problem of identifying whether an individual is a carrier of a specific gene or DNA marker (herein referred to collectively as “DNA of interest”), such as a gene associated with a particular disease or genetic trait. It then becomes necessary for the researcher or other interested party to determine, first, which individuals can or should undergo DNA testing to assist in answering that question, and secondly, if a specific individual is tested, what information of assistance that will provide. To date there has been no way to easily answer such questions or to permit individuals to interact as a group in assisting such research.

A currently popular application of genetic research is genetic genealogy. As genetic genealogy becomes more popular, an increasing number of people are requesting genetic testing to answer questions pertaining to their genealogy. A common obstacle encountered by individuals trying to utilize genetics to solve genealogical questions is the determination of which individual in the family tree needs to be tested to trace certain ancestral lines (e.g. who is the carrier of the DNA of interest to answer a specific genealogical question). Another common obstacle is the determination of which ancestor/ancestral line will be uncovered when testing a certain individual in the family tree.

When a male individual tests his Y-chromosome DNA (“Y-DNA”), he is testing only a single line, his paternal line (father's father's . . . line). When a male individual tests his mitochondrial DNA (“mtDNA”), he is testing only a single line, his maternal line (mother's mother's . . . line). A female individual does not have Y-DNA so she cannot test herself to trace her paternal line. However, female individuals do have mtDNA, so a female can test her mtDNA to trace her maternal line. Y-DNA and mtDNA testing only allows for tracing a single line in the family tree and will not address the ancestry of all of the lines in between (e.g. mother's father's ancestry, father's mother's ancestry, etc.). Many individuals are interested in tracing various or specific family lines, and can only do so by testing other relatives who are the direct carriers of the mtDNA or Y-DNA for those lines. Also, females may be curious about their own paternal line. In such situations, females must test another male family member who is a carrier of her father's Y-DNA in order to trace her own paternal ancestry. For example, a female may trace her paternal line by testing the Y-DNA of her father, or her brother, or her father's brother, etc. In another example, individuals interested in tracing the ancestry of their father's mother's maternal line must test the carrier of the mtDNA for this line, e.g. their father is a carrier, their father's mother is a carrier, all of their father's mother's children are carriers, and the children of their father's mother's daughters are carriers, etc. The questions that are posed regarding who needs to be tested in order to trace the ancestry of a particular ancestor or family line can become exceedingly complex, and there is currently no method for individuals to easily find out who in the family needs to be tested to uncover the ancestry of a line or ancestor in question.

Thus the two most common questions encountered by someone who is interested in using genetic genealogy to uncover a specific family line are: i) can the individual test himself/herself to trace the ancestry of the ancestor or ancestral line that he/she is interested in, and, if not, ii) who else in the family tree is the carrier of the DNA of interest and can be tested to answer the question?

In order to determine who is the carrier of the DNA of interest, that is, who needs to be tested in order to trace the ancestry of a specific ancestor or ancestral line, an understanding of the inheritance pattern of the DNA of interest is required to determine who should be tested to trace the line in question. However, for the layperson, this is often a tedious task and difficult. There is currently no easy way for individuals to jointly test themselves as well as others in the family to jointly uncover or resolve the ancestry of the various ancestors, lines and branches in the family tree.

Also, there is no method to accurately document, trace, store, view, compare and analyze the data for the various genealogical lines in the family which are tested, and no method to easily determine who should be tested to determine a particular line, and which line will be uncovered by testing a given individual. It is also common that individuals cannot determine which family line they will be tracing when testing a particular person in the family tree. For example, a female who is interested in tracing her paternal ancestry (her father's paternal line) will need to test her father or a male relative who carries her father's Y-DNA. Also, many individuals may be curious about the ancestry of a particular ancestor. For example an individual may have heard tales that her great grandmother (mother's father's mother) is native, but does not know which living relative carries the DNA of interest required to trace or confirm the ancestry of this ancestor.

Each individual has a large number of ancestors and ancestral lines, but they themselves are usually only carriers of a single Y-DNA line (if male) and a single mtDNA line (male or female). In order to find out the ancestry of the rest of their ancestors and lines, they will need to test the correct individual in their family tree who is a direct carrier of the DNA of interest. This is often a daunting task and is a major obstacle for individuals wishing to use Y-DNA or mtDNA to trace their ancestry.

The present inventors have disclosed in co-pending U.S. patent applications Ser. Nos. 11/382,430 filed May 9, 2006 and 11/552,933 filed Oct. 25, 2006, which are incorporated herein by reference, methods of constructing a family tree using an online social network.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

The invention provides a method and system for conducting genetic research using DNA sampling, preferably via an online social network, using a set of family members related by defined genealogical relationships to identify carriers of a DNA of interest for obtaining DNA information. According to various aspects of the invention, the method can be used for identifying and determining carriers of a DNA of interest, and further genealogical analysis.

More particularly, the invention provides a computer-implemented method of conducting genetic investigation using a set of individuals related by defined genealogical relationships, comprising: a) defining a set of living or deceased individuals related by defined genealogical relationships; b) defining at least one DNA type of interest; c) defining the inheritance pattern associated with each said at least one DNA type of interest; d) determining the information associated with each said at least one DNA type of interest; e) obtaining information in relation to said set of living or deceased individuals, by carrying out an inquiry using the information obtained in at least one of steps a), b), c) or d). The inquiry may be selected from the group consisting of: i) selecting a DNA type of interest and determining which information can be obtained by testing the DNA type of interest; ii) selecting a DNA type of interest and determining which individual in the set carries said DNA type of interest; iii) selecting a DNA type of interest and determining which ancestral/family line can be traced by testing said DNA type of interest; iv) selecting an individual from said set and determining which DNA type is carried by the individual; v) selecting an individual from the set and determining which ancestral/family line can be traced by testing the DNA types carried by the individual; vi) selecting an individual from the set and determining which ancestral/family line can be traced by testing a DNA type of interest; vii) selecting an individual from the set and determining which ancestral/family line can be traced by testing each DNA type carried by the individual; viii) selecting a first individual from the set and determining which additional individuals in the set belong to the same ancestral/family line as the first individual; ix) selecting a first individual from the set and determining which additional individual in the set carries the same DNA characteristic for the DNA type of interest as said first individual; x) selecting a first individual from the set and determining which other individuals in the set carry DNA which can provide information for the individual; xi) selecting a first individual from the set and determining which information for which other individual from the set can be derived from testing the first individual; xii) selecting a first individual from the set, selecting a second individual from the set and determining which DNA type has the same DNA characteristic for the first individual and the second individual; xiii) selecting a first individual from the set, selecting a second individual from the set, selecting at least one DNA type and determining whether the DNA characteristic for the at least one DNA type is the same for the first individual and the second individual; xiv) selecting an individual from the set, selecting a DNA type and determining whether the individual carries said DNA type; xv) selecting an individual from the set, selecting a DNA type and determining whether the individual can be tested for said DNA type; xvi) selecting a first individual from the set, selecting a DNA type and determining which additional individual carries the same DNA characteristic for the DNA type as the first individual; xvii) selecting an individual from the set, selecting a DNA type and determining which ancestral/family lineage is traced by testing the individual and the DNA type; xviii) selecting an individual from the set, selecting ancestral/family line for investigation, determining whether the individual carries the DNA type required to trace the ancestral/family line; xix) selecting a first individual from the set, selecting an ancestral/family line, determining which additional individual is in the same ancestral/family line as the first individual; xx) selecting an ancestral/family line of interest and determining which individual in the set carries the DNA type required to trace said ancestral/family line; and xxi) selecting an ancestral/family line and determining which individual and which DNA type is required to trace the ancestral/family line.

One aspect of the preferred method uses a computer system to identify one or more than one carrier of a particular DNA of interest, determine in a family tree the carriers of the DNA of interest who can contribute information through DNA testing, determine what information the testing of an individual in the tree will contribute, communicate with carriers so identified and recruit such carriers for such DNA testing, order the sampling, conduct research, and add the information to the family tree in order to document, store, view, compare and analyze the data for the various genealogical lines in the family which are tested.

The current invention makes use of a computerized network to allow one or more users of the system to interactively and jointly uncover the various lines in their family tree. Such a family tree, in the preferred state, is in the form of an online social network which allows users and/or family members to interactively and jointly test, uncover, share and view one or more lines of the family tree. The ability of family members to jointly and interactively work together to test, identify and recruit necessary individuals for testing, viewing, analyzing, inviting and communicating through the system allows people to more easily make use of the powerful capabilities of genetic genealogy to unravel their ancestry.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIG. 1 is a schematic family tree diagram illustrating the Y-chromosome DNA pattern of inheritance;

FIG. 2 is a schematic family tree diagram illustrating the mitochondrial DNA pattern of inheritance;

FIG. 3 is a schematic family tree diagram;

FIG. 4 is a flowchart illustrating one aspect of the invention.

FIG. 5 is a detailed flowchart illustrating one aspect of the invention.

FIG. 6 is a schematic family tree diagram illustrating the nodes and genetic lines in a family tree.

FIG. 7 is a flowchart illustrating a further aspect of the invention.

FIG. 8 is a flowchart illustrating a further aspect of the invention.

FIG. 9 is a flowchart illustrating a further aspect of the invention.

FIG. 10 is a flowchart illustrating a further aspect of the invention.

FIG. 11 a flowchart illustrating a further aspect of the invention.

FIG. 12 a flowchart illustrating a further aspect of the invention.

FIG. 13 is a schematic drawing illustrating a computer network implementing a social network according to the invention.

### DESCRIPTION

Throughout the following description, specific details are set forth in order to provide a more thorough understanding to persons skilled in the art. However, well known elements may not have been shown or described in detail to avoid unnecessarily obscuring the disclosure.

Accordingly, the description and drawings are to be regarded in an illustrative, rather than a restrictive, sense.

In the following description, “DNA type of interest” can be any specific DNA construction, whether an entire chromosome or specific markers in the DNA such as defined short tandem repeat (STR) markers, single nucleotide polymorphism (SNP) markers, deletions, insertions, mutations or a gene. Examples of the information associated with a DNA type of interest are the inheritance pattern (e.g. Y-DNA is passed down from father to son along the paternal line), carriers and associations (e.g. Y-DNA is carried only by males, Y-DNA haplogroup H subclade SNPs are only carried by individuals who belong to haplogroup H, etc.). The “same DNA characteristic for said DNA type of interest” means a characteristic of the DNA is identical as between two instances of a DNA type of interest. For example, where the DNA type of interest is Y-DNA, and two individuals have the same Y-DNA characteristic, they have the same DNA characteristic for said DNA type of interest. For example a father and son have the same Y-DNA characteristic. “Ancestral/family line” or “family line” means a continuous line of ancestry or descent. “Carrier” means an individual who carries a specific instance of a DNA type of interest.

In the following description the method is described using a family tree as the preferred form of ordered set of family relationships. The method is also useful where the ordered set is not strictly displayed to the user as a family tree, but merely stored, such as in computer memory or a computer database, as a list or collection of data, such as names of family members with accompanying metadata, list or index specifying the relationships between individual, whether living or deceased, and the like. Also in the preferred embodiment users interact with the family tree via an online social network. However for purposes of the invention it is merely necessary that users of the system communicate with he system by any means of communication, whether electronic, telecommunication, written, verbal or otherwise. Details of examples of “online social networks” are known in the art and are described in the applicant's co-pending U.S. patent application Ser. Nos. 11/382,430 filed May 9, 2006, and 11/552,933 filed Oct. 25, 2006, which are incorporated herein by reference.

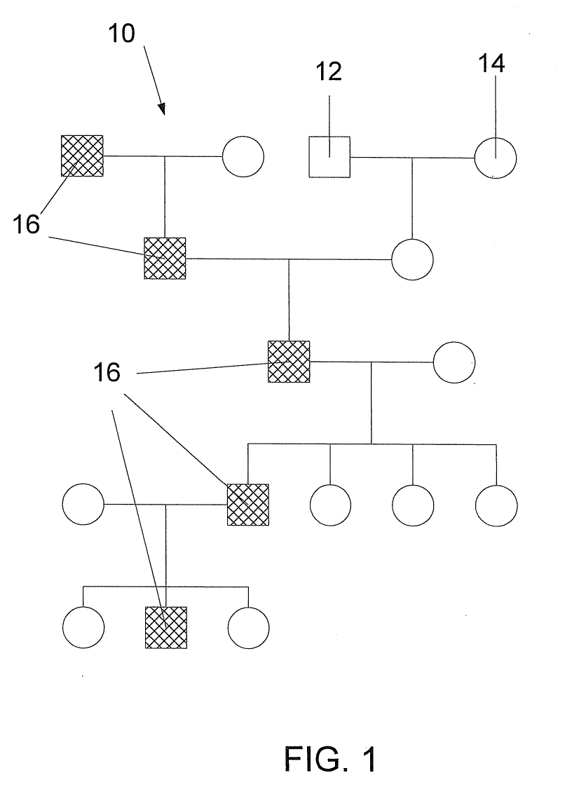

With reference to FIG. 1, a simplified family tree 10 wherein squares 12 represent males and circles 14 represent females illustrates the Y-DNA pattern of inheritance. It is well known that male offspring inherit the entire Y-chromosome from their father, and that both male and female progeny inherit their mother's entire mtDNA. Therefore the haplotype or haplogroup of an individual's Y-chromosome is a relatively constant characteristic of the Y-DNA which can be used to thereby link an individual to an ancestor through a series of direct male progenitors. FIG. 1 shows as cross-hatched squares the carriers 16 which carry the same line of Y-chromosome (haplotype) which is passed only from father to son. Thus if any one of carriers 16 has a Y-chromosome identified by a particular marker or haplotype then it is known that all the other carriers 16 have the same line and are direct male descendants or ancestors of that carrier. Conversely if a male is a direct male descendant or ancestor of another male carrier of a particular Y-chromosome line then it is known that that male also has the same Y-chromosome. The series of male carriers of the same Y-chromosome line is referred to herein as the paternal line, and each such carrier is referred to as a carrier of that paternal line.

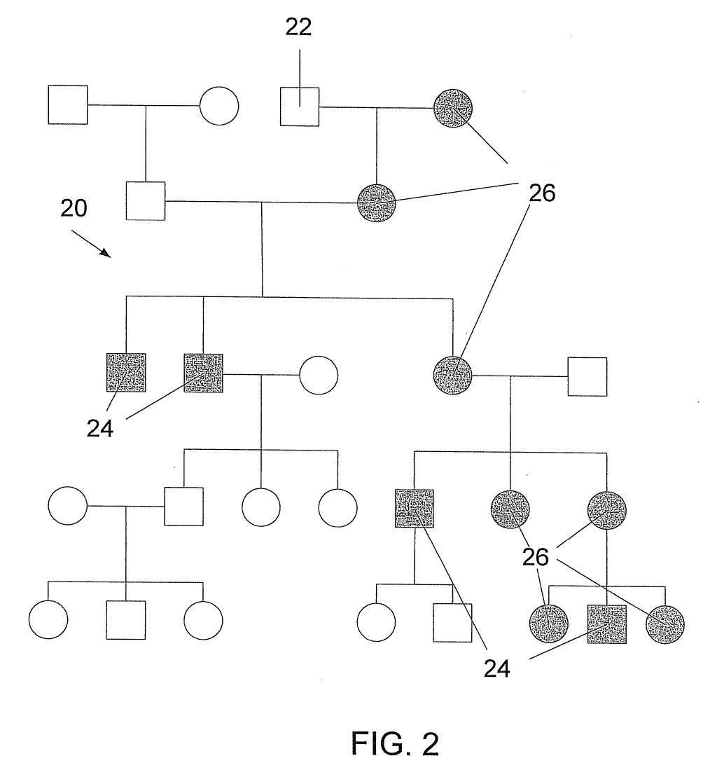

FIG. 2 illustrates a simplified family tree 20 wherein squares 22 represent males and circles 24 represent females, showing the mtDNA pattern of inheritance. The mtDNA is passed to male and female progeny only by the mother. MtDNA can thus be used to link an individual to a female ancestor through a series of male and female progenitors. FIG. 2 shows as shaded squares and circles 24, 26 the carriers which carry the same mtDNA which is passed to male and female progeny only by the mother. Thus if any one of carriers 24, 26 has a mtDNA identified by particular markers then it is known that all the other carriers 24, 26 have the same line and are descendants of a common maternal carrier of that line. Conversely if a male or female is a direct descendant through females of a female carrier of a particular mtDNA then it is known that that male or female also has the same mtDNA. The series of male and female carriers of the same mtDNA is referred to herein as the maternal line, and each such carrier is referred to as a carrier of that maternal line.

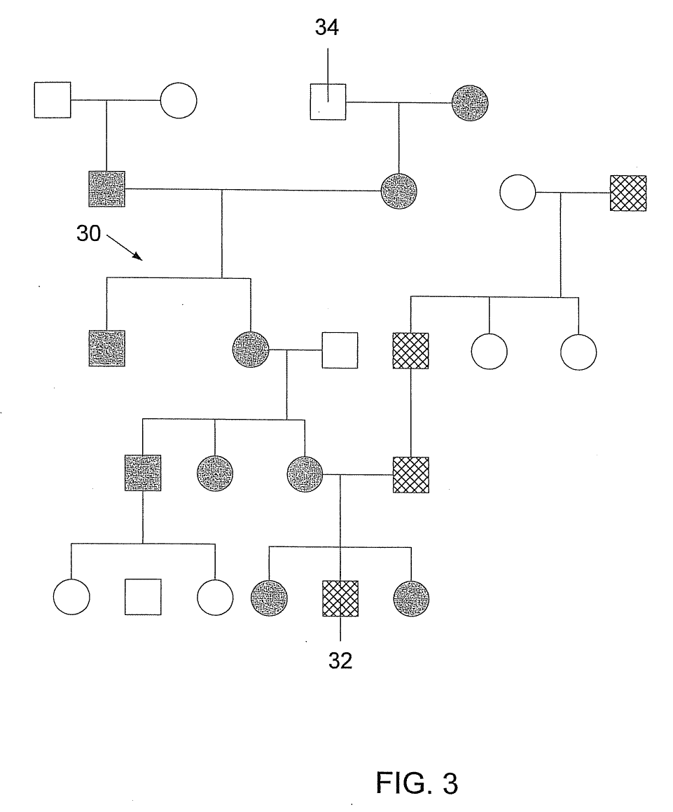

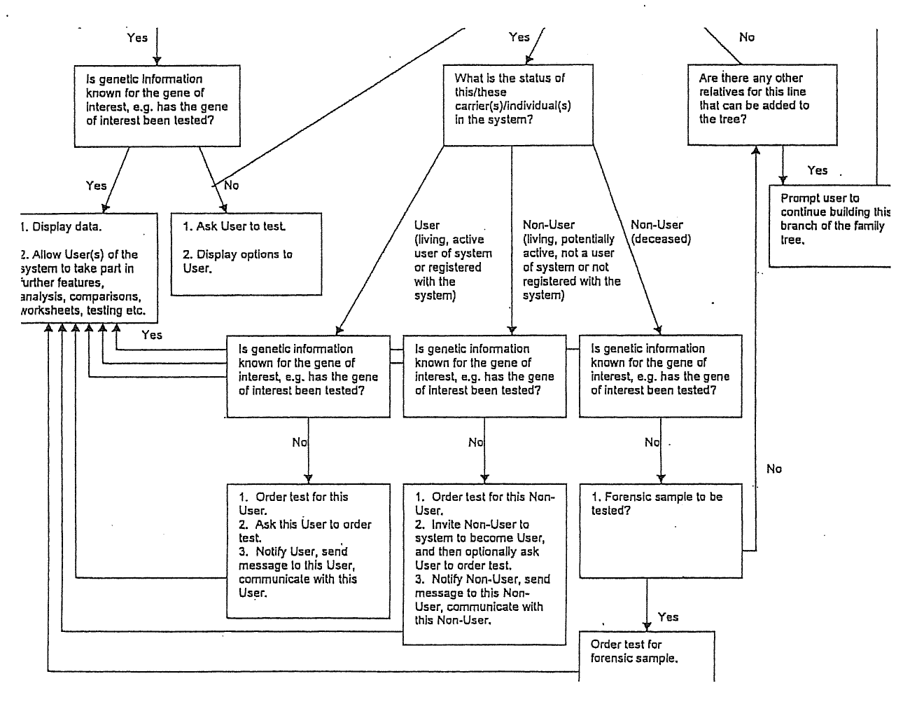

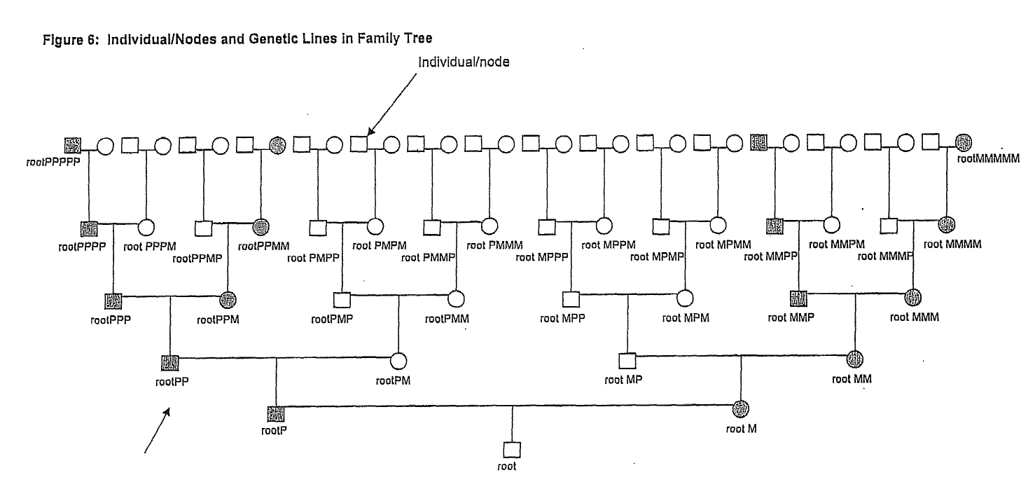

In order to implement the invention, the system generates initially a family tree, as shown in FIGS. 3 and 6. The term “family tree” is defined herein to include not only the visual or graphic representation of a family tree as shown in FIGS. 3 and 6, but in general any ordered collection of data representing the relationships of individuals in a family. For example, a “family tree” may be a network of nodes and linkages representing the relationships of individuals in a family. To illustrate the method of the invention, a partial family tree 30 for an individual user 32 has been prepared by the system as shown in FIG. 3. Individual user 32 wishes to trace the ancestry of ancestor 34. The system, as further described below, maintains one or more family trees and associated data, tools and features, and is accessible to one or more users. While the invention has application to identify and locate carriers of any DNA of interest having various inheritance patterns, FIG. 6 illustrates the application of the method to paternal Y-DNA and maternal mtDNA lines. With reference first to FIG. 6, a simplified partial family tree is illustrated in which squares represent male individuals and circles represent female individuals. The root male is shown at the base of the tree as “root”. A node on any given level is linked by a line to its mother and father, joined by a horizontal line in the level above it. In the following description, a genetic line consists of a number of individuals in the family tree who share a common DNA of interest, such DNA of interest having a known inheritance pattern. Examples are strict maternal line inheritance such as mtDNA, strict paternal line inheritance such as Y-DNA, etc. In FIG. 6, root=root of tree, P=Paternal/Father, M=Maternal/Mother. Thus for example, rootPMPM designates the root's father's mother's father's mother. A “carrier” is an individual who possesses or is in any way affected by the DNA of interest. In one embodiment of the invention, the DNA of interest are the Y-chromosome DNA which defines a paternal line of inheritance and the mtDNA which defines a maternal line of inheritance, but other DNA or genes of interest with defined method of inheritance will work in the invention, such as, for example, the genetic marker for hemophilia. In FIG. 6, the paternal Y-DNA line of the root are the shaded squares rootP, root PP etc. The paternal Y-DNA line of the rootNM are the shaded squares rootMMP, rootMMPP etc. The maternal mtDNA line of the rootPP are the shaded squares rootPPM, rootPPMM etc.

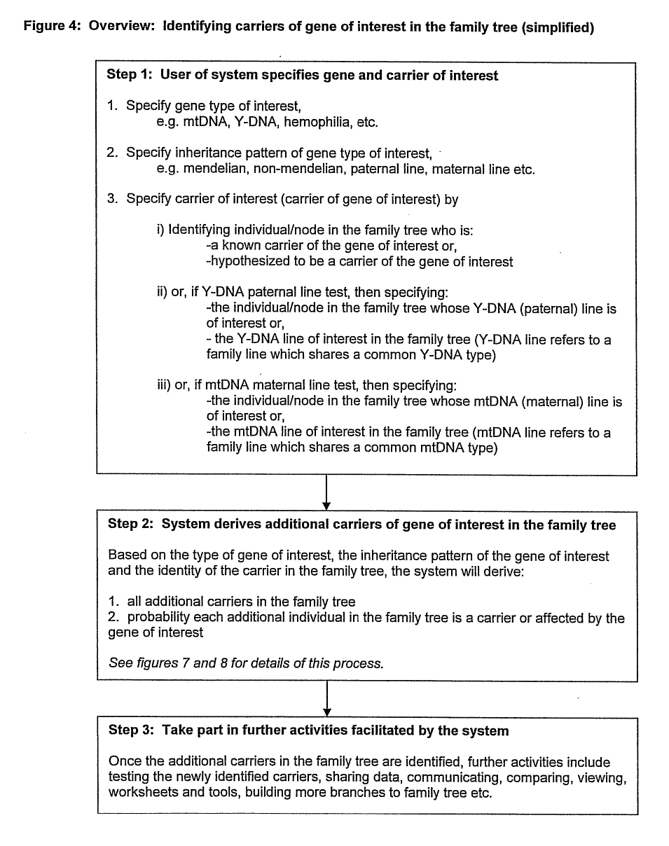

FIG. 4 is a flowchart providing a simplified overview outlining the use of the system to specify the DNA of interest and the use of the system to present to the user the individuals in the family tree who carry the DNA of interest and who can or could be tested to answer the question of interest to the user. Once the carrier of the DNA of interest is identified, the user can further use the system to take part in activities facilitated by the social networking capabilities of the system to share, store, view and conduct research alone or jointly with other users. Referring to the flowchart in FIG. 4, the flowchart illustrates in a simplified way how the method identifies carriers of the DNA of interest in the family tree. The user of the system specifies the DNA type of interest, e.g. mtDNA, Y-DNA, hemophilia, etc. (Where the following steps are described as being done by a user, such user may be an individual registered as part of the system or not, or the system itself on behalf of a party.) For each DNA type, the inheritance pattern of the DNA type of interest may be determined or already associated with it, e.g. mendelian, non-mendelian, paternal line, maternal line etc. A carrier of the DNA of interest is selected by i) identifying an individual/node in the family tree who is either a) a known carrier of the DNA of interest or, b) hypothesized to be a carrier of the DNA of interest; or ii) if a Y-DNA paternal line test is being performed, then specifying a) the individual/node in the family tree whose Y-DNA (paternal) line is of interest or, b) the Y-DNA line of interest in the family tree (Y-DNA line refers to a family line which shares a common Y-DNA type); or iii) if an mtDNA maternal line test is being performed, then specifying a) the individual/node in the family tree whose mtDNA (maternal) line is of interest or, b) the mtDNA line of interest in the family tree (mtDNA line refers to a family line which shares a common mtDNA type).

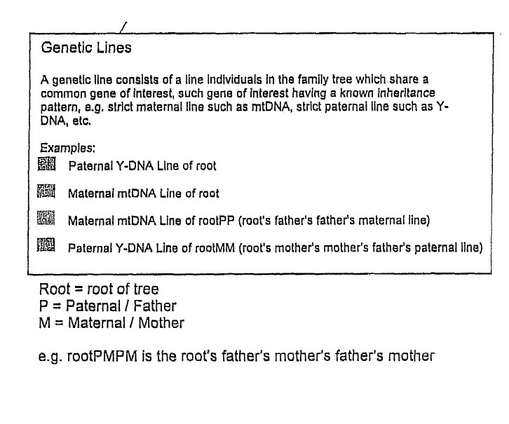

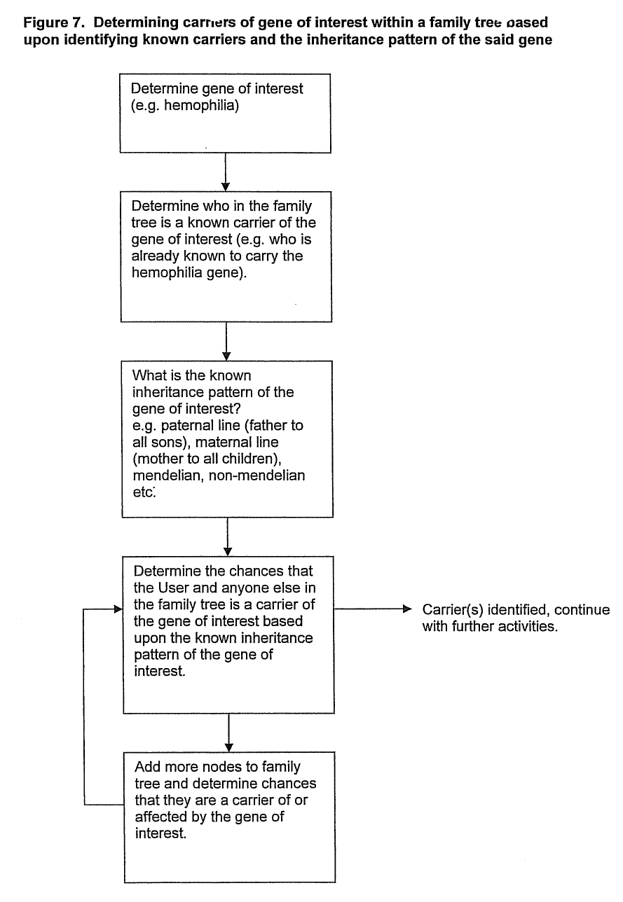

The system determines additional carriers of the DNA of interest in the family tree. Based on the type of DNA of interest, the inheritance pattern of the DNA of interest and the identity of the carrier in the family tree, the system will derive: 1) all additional carriers of the DNA of interest in the family tree; and 2) the probability that each additional individual in the family tree is a carrier or affected by the DNA of interest. FIGS. 7 through 11 provide details of this process for paternal and maternal line analyses. In Step 3, once the additional carriers in the family tree are identified, the system facilitates further activities including testing the newly identified carriers, sharing data, communicating, building more branches to the family tree, etc.

Some of the general benefits of this method include: i) Automatic identification of the distribution of the DNA of interest in the family tree (identification of carriers); ii) Automatic identification of the risks and probability that a member of a family will carry or become affected by a DNA of interest; iii) Automatic identification of individuals who needs to be tested to answer a genetic question; iv) Automatic identification of individuals who are carriers of the DNA of interest and the probability that they may be affected; v) Automatic identification of which DNA of interest an individual carries; vi) Automatic identification of which line will be uncovered when a selected individual from the family tree is tested; vii) The system allows people in the family tree to be contacted for testing, sharing information and communication; and viii) The system provides a common place to record, store and display data by individual or by line.

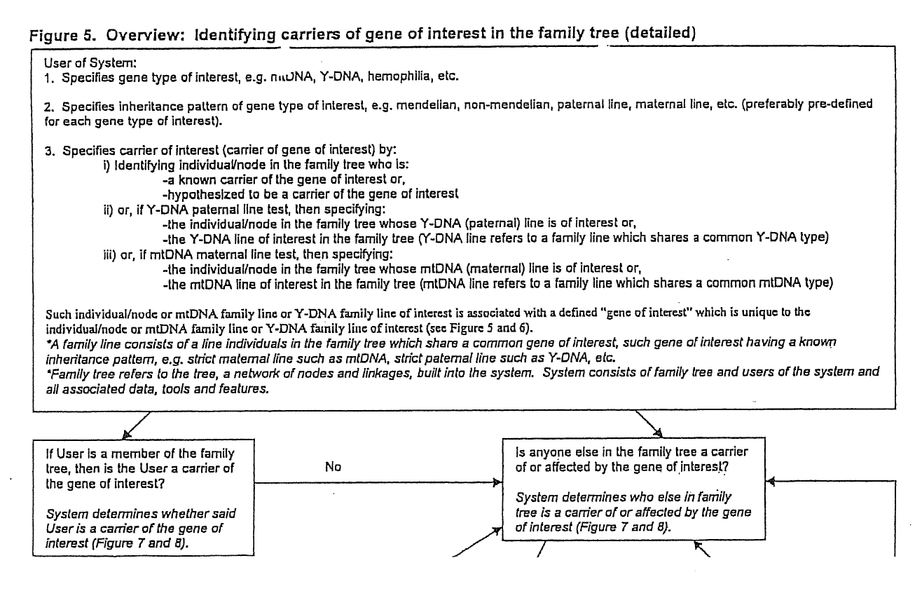

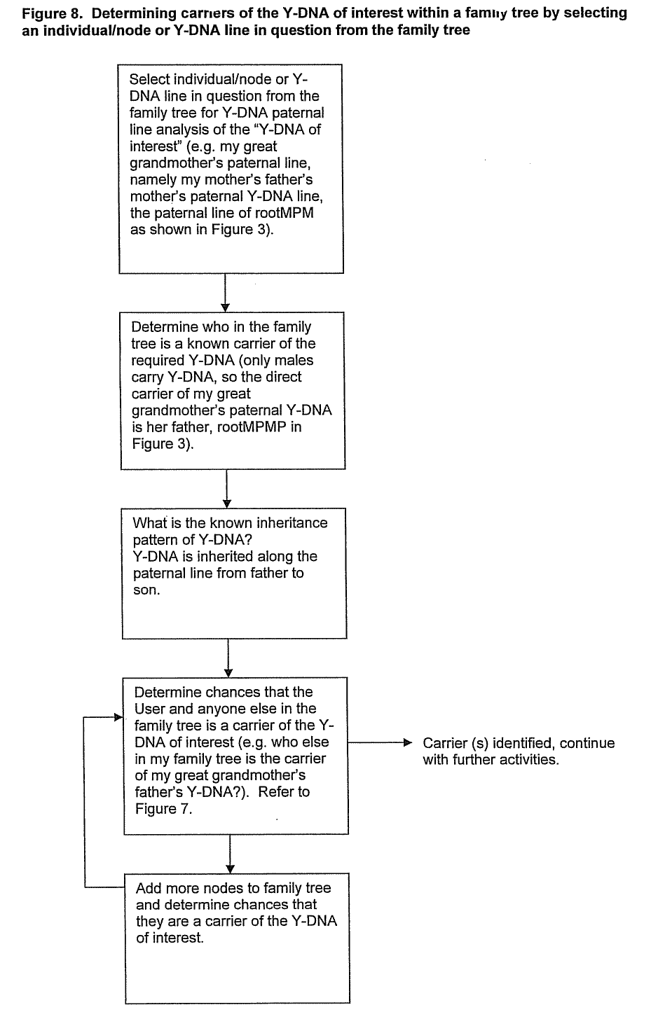

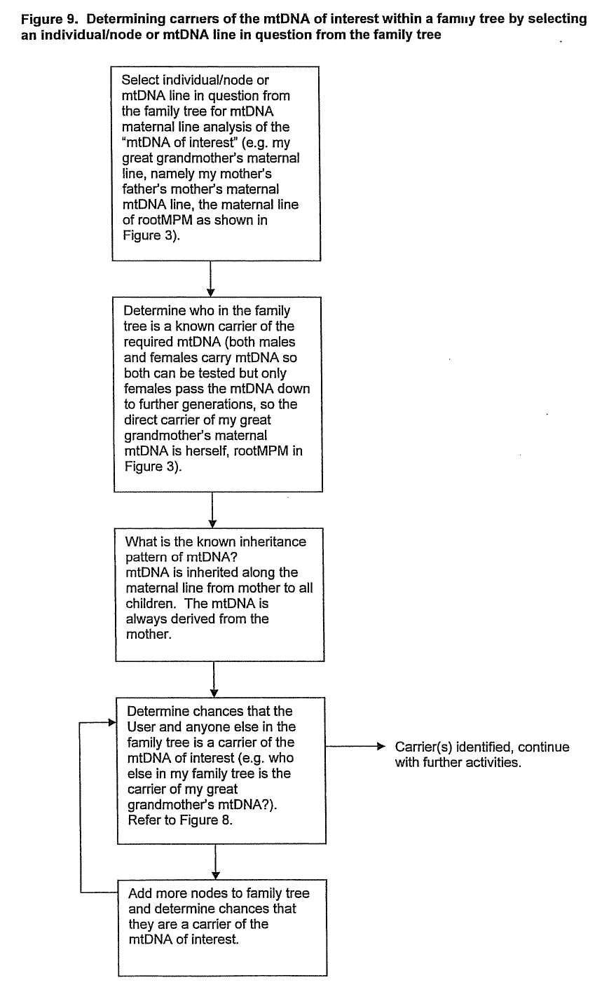

FIG. 5 is a detailed overview outlining one aspect of the invention which describes the use of the system to specify the line desired to be traced. The system informs the user whether the user himself/herself can be tested or carries the DNA of interest, or whether anyone else in the family tree can be tested. If the user can be tested, the user can use the system to order a test, or if the results are already known, the user can use the system to view the results and continue with further activities. The system will also notify the user whether someone else in the family tree can be tested to provide results to the line in question. If not, the system will prompt the user to build more individuals into the required line of the tree and the system will determine which newly added individual can be tested or is a carrier of the DNA of interest. Once one or more carriers are identified, the system will display the status of the user in the system. Referring to the flowchart in FIG. 5, the flowchart illustrates in more detail how the method identifies carriers of the DNA of interest in the family tree. The user of the system specifies a DNA type of interest, e.g. mtDNA, Y-DNA, hemophilia, etc. The user determines the inheritance pattern of the DNA type of interest, e.g. mendelian, non-mendelian, paternal line, maternal line etc. Preferably the inheritance pattern is pre-defined for each DNA type of interest. A carrier of the DNA of interest is selected by: i) identifying an individual/node in the family tree who is: a) a known carrier of the DNA of interest or, b) hypothesized to be a carrier of the DNA of interest; or ii) if a Y-DNA paternal line test is being performed, then specifying: a) the individual/node in the family tree whose Y-DNA (paternal) line is of interest or, b) the Y-DNA line of interest in the family tree (Y-DNA line refers to a family line which shares a common Y-DNA type); or iii) if an mtDNA maternal line test is being performed, then specifying: a) the individual/node in the family tree whose mtDNA (maternal) line is of interest or, b) the mtDNA line of interest in the family tree (mtDNA line refers to a family line which shares a common mtDNA type). As further illustrated in FIGS. 8 and 9, such individual/node or mtDNA family line or Y-DNA family line of interest is associated with a defined DNA of interest which is unique to the individual/node or mtDNA family line or Y-DNA family line of interest. A family line consists of a line of individuals in the family tree which share a common DNA of interest, such DNA of interest having a known inheritance pattern, e.g. strict maternal line such as mtDNA or strict paternal line such as Y-DNA.

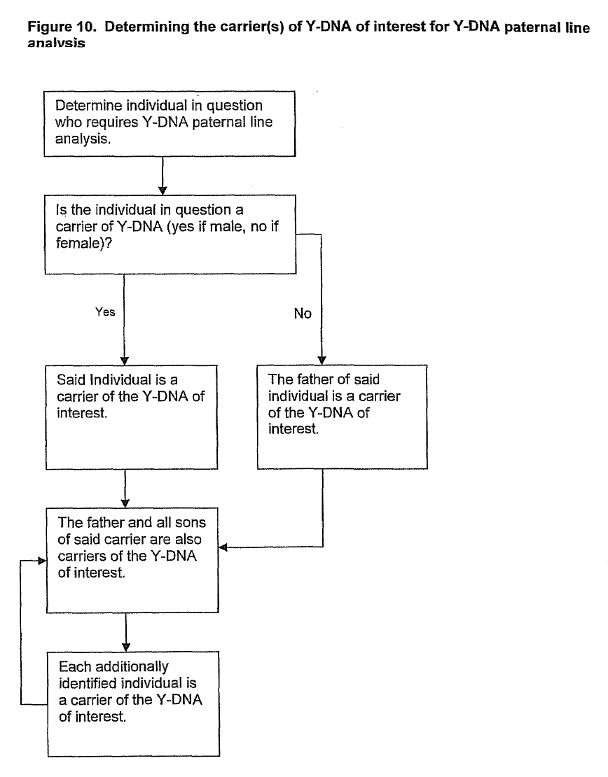

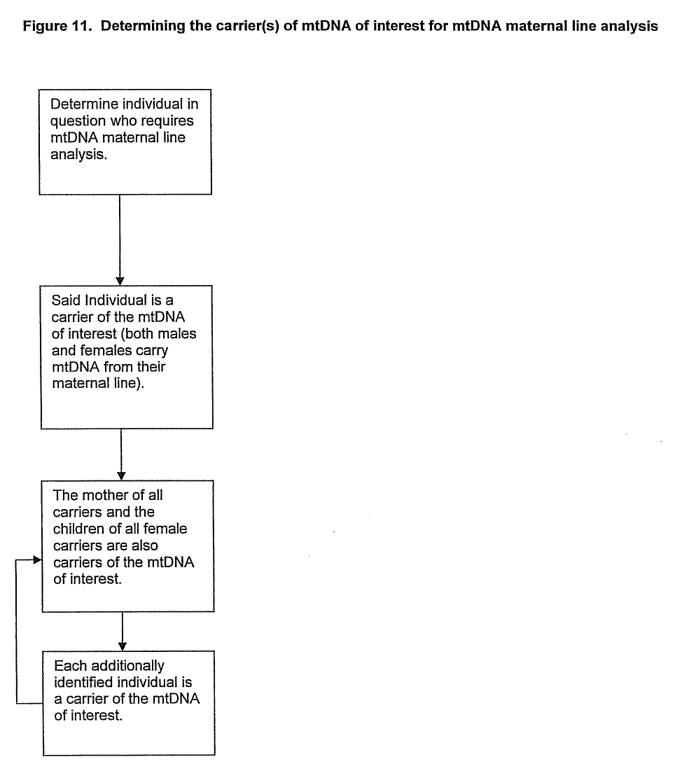

If the user is a member of the family tree, the system inquires whether the user is a carrier of the DNA of interest. If he or she is, and the genetic information for the user's DNA of interest is known from previous testing, then the data is displayed to the user and the user is permitted to take part in further features, analysis, comparisons, worksheets, testing and the like. If not, then the user is asked to test his/her DNA and options for doing so are displayed to the user. The system also determines if anyone else listed in the family tree is a carrier of or affected by the DNA of interest. This is determined based on the biological relationship of each other person in the family tree, according to the selected DNA of interest, as further illustrated in FIGS. 10 and 11. If an individual is a carrier, then the system determines the status of the carrier, whether another user (living, active user of the system or registered with the system), non-user (a living and potentially an active user but not currently a user and not registered with the system) or deceased. For active users, if the user's genetic information for the DNA of interest is known through previous testing, then the data is displayed to the user and the user is permitted to take part in further features, analysis, comparisons, worksheets, testing and the like. If not, a DNA test is sought by the system for the user. In particular, either a test is ordered or the user is asked to order a DNA test and the user is notified, by sending an email message or other form of communication. For a living non-user, if the person's genetic information for the DNA of interest is known through previous testing, then the data is displayed to the user and the user is permitted to take part in further features, analysis, comparisons, worksheets, testing and the like. If not, the system seeks to order a DNA test for the non-user by ordering the test or by inviting the non-user to become a user of the system, then once the person has become a user asking him/her to optionally order a DNA test. Again the user can be notified, by sending an email message or other form of communication. If the individual is deceased, then the system inquires if the deceased's genetic information is known for the DNA of interest through previous testing. If so, then the data is displayed to the user and the user is permitted to take part in further features, analysis, comparisons, worksheets, testing and the like. If not, the system asks whether a forensic sample can be obtained. If so, a forensic DNA test is ordered. If not the system inquires whether there are any other relatives from this line that can be added to the tree. If so, the user is prompted to continue building this branch of the tree and the process repeated for the new family members. The system examines each person in the tree who is a carrier.

FIG. 7 illustrates in general how a carrier of the DNA of interest is determined within a family tree by identifying known carriers and the inheritance pattern of the gene. The DNA of interest is determined (for example hemophilia). It is determined who in the family tree is a known carrier of the DNA of interest. The rules of the inheritance pattern for the DNA of interest are determined. The likelihood that the user and anyone else in the family tree is a carrier of the DNA of interest, based upon the known inheritance pattern of the DNA of interest is determined. More nodes are added to the family tree and the foregoing step is repeated. Once the carriers are identified, the process in FIG. 7 is continued. FIG. 8 illustrates the general method shown in FIG. 7 where the selected genetic line is Y-DNA. FIG. 9 illustrates the general method shown in FIG. 7 where the selected genetic line is mtDNA.

FIG. 10 illustrates how the carriers of the Y-DNA of interest are determined for a paternal line analysis. It is determined who the individual is in the family tree who requires the Y-DNA of interest for a paternal line analysis. If the individual is a female, the father of the individual is a carrier. If the individual is a male, that individual will always be a carrier of the Y-DNA of interest. The father and all sons of all such carriers are then designated as carriers of the Y-DNA of interest. When each additional carrier is identified, the foregoing step is repeated until all individuals in the family tree have been assessed.

FIG. 11 illustrates how the carriers of the mtDNA of interest are determined for a maternal line analysis. It is determined who the individual is in the family tree who requires the mtDNA maternal line analysis. That individual will always be a carrier of the mtDNA of interest. The mother of all carriers and all children of female carriers are then designated as carriers of the mtDNA of interest. When each additional carrier is identified, the foregoing step is repeated until all individuals in the family tree have been assessed.

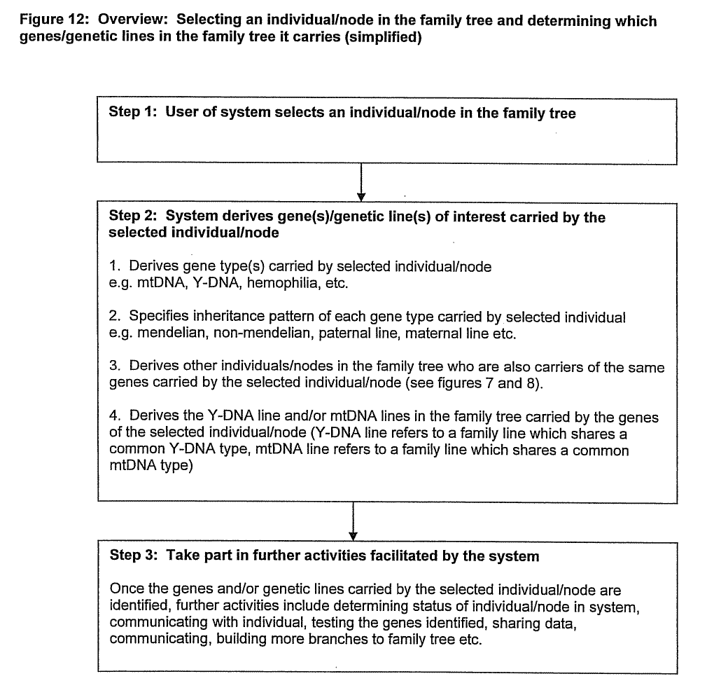

FIG. 12 illustrates generally in simplified form the steps in the method for selecting an individual/node in the family tree and determining which DNA of interest/genetic lines in the family tree it carries. The user of the system selects an individual/node in the family tree. The system derives one or more than one DNA of interest/genetic line of interest carried by the selected individual/node e.g. mtDNA, Y-DNA, hemophilia, etc. The inheritance pattern of each gene type carried by selected individual e.g. mendelian, non-mendelian, paternal line, maternal line etc. is then determined. The system derives other individuals/nodes in the family tree who are also carriers of the same genes carried by the selected individual/node as described in FIGS. 8 and 9. The system derives the Y-DNA line and/or mtDNA line in the family tree carried by the DNA of interest of the selected individual/node (Y-DNA line refers to a family line which shares a common Y-DNA type, mtDNA line refers to a family line which shares a common mtDNA type). Once the DNA of interest and/or genetic lines carried by the selected individual/node are identified, further activities include testing the DNA of interest identified, sharing data, communicating, building more branches to family tree etc.

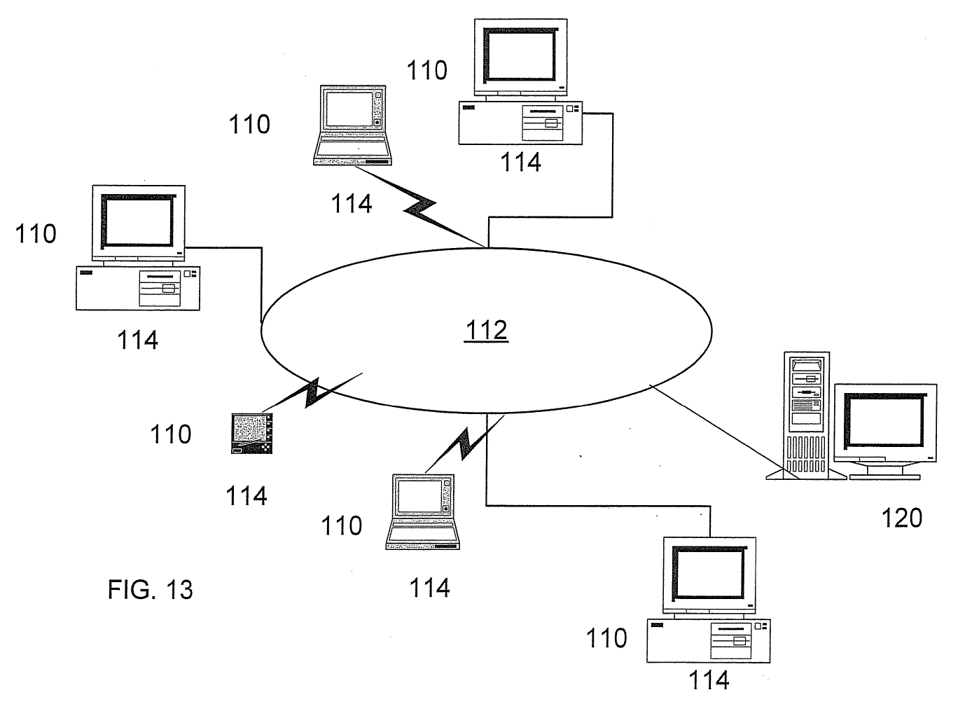

With reference to FIG. 13, in the preferred form of the invention the system comprises a social network accessible to a number of users 110 having computer terminals 114, whether laptops, desktops, handheld, personal digital assistants, mobile telephones or any other device capable of communicating over a network, via a computer network 112 such as the Internet, whether wirelessly or through wired access. Terminals 114 may have an internet browser and an e-mail application for example, and may themselves include a local or wide area network. A social network server or servers 120 maintain a system comprising an Internet website, database and associated data, which is accessible to users 110. Server 120 has the hardware and software required to operate an internet-accessible social network, including web server, email server, databases, data storage and the like.

Initially user 110 accesses the system website and is presented with a page to register for the system by entering basic profile information, such as name, gender, address, birthdate and a username and password. A unique identifier, such as a number, alphanumeric or alphabetic string, is assigned to the user and, once registered, a representation or node in the system, is created for the user. All of the foregoing are carried out in a manner well known to web site programmers. Such node is designated “active” as previously indicated.

Each active node can contain information pertaining to the living individual person that it represents, communication tools, data and files, storage space, network information and tools to edit the network of living and non-living entities. Nodes can contain the genetic information. Each active node allows the individual person that it represents to log into the system and access its own node and its associated “control panel”. The user can upon registration and thereafter, carry out a number of tasks or activities while at the “control panel” of his/her node, including 1) edit his/her own node; 2) build a network of living and deceased individuals (hereinafter “the network”); 3) edit the network; 4) communicate with other entities within or outside the network using the features of the social network; and 5) make use of projects and features facilitated by the network of living and non-living entities. The control panel allows an individual person to i) expand and edit the network of living and deceased individuals by: adding nodes and linkages; editing nodes and linkages; inviting other individual persons to join; and ii) communicate with nodes in the same network, other nodes in the computerized network, as well as other living individual persons outside the network; send invitations; accept invitations; grant permissions; share information; compare information; and make use of the projects and features facilitated by the network. Other features of such a social network are as described in co-pending U.S. patent application Ser. Nos. 11/382,430 filed May 9, 2006, and 11/552,933 filed Oct. 25, 2006, which are incorporated herein by reference.

The system described above therefore facilitates many form of genealogical inquiry. For example a user may choose one of his or her ancestors and inquire of the system of the user's family members are carriers of DNA markers that will provide information relative to that ancestor. Or the user may want to identify the line which will provide certain information concerning the ancestor. Or the user may want to know which of his or her relatives' DNA can be tested to provide certain information. Or the user may want to know if a particular relative's DNA is tested, what information would be provided. Or the user may want to know all the relatives whose DNA can be tested and what information would be revealed.

The system facilitates the following inquiries: i) selecting a DNA type of interest and determining which information can be obtained by testing the DNA type of interest; ii) selecting a DNA type of interest and determining which individual in the set carries said DNA type of interest; iii) selecting a DNA type of interest and determining which ancestral/family line can be traced by testing said DNA type of interest; iv) selecting an individual from said set and determining which DNA type is carried by the individual; v) selecting an individual from the set and determining which ancestral/family line can be traced by testing the DNA types carried by the individual; vi) selecting an individual from the set and determining which ancestral/family line can be traced by testing a DNA type of interest; vii) selecting an individual from the set and determining which ancestral/family line can be traced by testing each DNA type carried by the individual; viii) selecting a first individual from the set and determining which additional individuals in the set belong to the same ancestral/family line as the first individual; ix) selecting a first individual from the set and determining which additional individual in the set carries the same DNA characteristic for the DNA type of interest as said first individual; x) selecting a first individual from the set and determining which other individuals in the set carry DNA which can provide information for the individual; xi) selecting a first individual from the set and determining which information for which other individual from the set can be derived from testing the first individual; xii) selecting a first individual from the set, selecting a second individual from the set and determining which DNA type has the same DNA characteristic for the first individual and the second individual; xiii) selecting a first individual from the set, selecting a second individual from the set, selecting at least one DNA type and determining whether the DNA characteristic for the at least one DNA type is the same for the first individual and the second individual; xiv) selecting an individual from the set, selecting a DNA type and determining whether the individual carries said DNA type; xv) selecting an individual from the set, selecting a DNA type and determining whether the individual can be tested for said DNA type; xvi) selecting a first individual from the set, selecting a DNA type and determining which additional individual carries the same DNA characteristic for the DNA type as the first individual; xvii) selecting an individual from the set, selecting a DNA type and determining which ancestral/family lineage is traced by testing the individual and the DNA type; xviii) selecting an individual from the set, selecting ancestral/family line for investigation, determining whether the individual carries the DNA type required to trace the ancestral/family line; xix) selecting a first individual from the set, selecting an ancestral/family line, determining which additional individual is in the same ancestral/family line as the first individual; xx) selecting an ancestral/family line of interest and determining which individual in the set carries the DNA type required to trace said ancestral/family line; and xxi) selecting an ancestral/family line and determining which individual and which DNA type is required to trace the ancestral/family line.

The genetic testing carried out by the system can involve at least one of the following steps: a) identifying the individual for genetic testing; b) identifying the DNA type for genetic testing; c) identifying the availability of the said individual for genetic testing; d) identifying the ability to sample DNA from the said individual for genetic testing; e) identifying the ability to obtain a sample containing the DNA from the said individual for genetic testing; f) ordering a genetic test for the said individual for at least one DNA type; or g) obtaining a genetic test for an individual in the system.

While a number of exemplary aspects and embodiments have been discussed above, those of skill in the art will recognize certain modifications, permutations, additions and sub-combinations thereof. For example, while the preferred form of the invention is described as an online computer network, the invention also has application to local and wide area computer networks, intranets and stand-alone computer systems. It is therefore intended that the invention be interpreted to include all such modifications, permutations, additions and sub-combinations as are within its true spirit and scope.

What is claimed is:

1. A computer-implemented method of conducting genetic investigation using a set of individuals related by defined genealogical relationships, comprising:
a) defining a set of living or deceased individuals related by defined genealogical relationships;
b) defining at least one DNA type of interest;
c) defining the inheritance pattern associated with each said at least one DNA type of interest;
d) determining the information associated with each said at least one DNA type of interest;
e) obtaining information in relation to said set of living or deceased individuals, by carrying out an inquiry using the information obtained in at least one of steps a), b), c) or d).

2. The method of claim 1 wherein said inquiry is selected from the group consisting of:
i) selecting a DNA type of interest and determining which information can be obtained by testing said DNA type of interest;
ii) selecting a DNA type of interest and determining which individual in said set carries said DNA type of interest;
iii) selecting a DNA type of interest and determining which ancestral/family line can be traced by testing said DNA type of interest;
iv) selecting an individual from said set and determining which DNA type is carried by said individual;
v) selecting an individual from said set and determining which ancestral/family line can be traced by testing the DNA types carried by said individual;
vi) selecting an individual from said set and determining which ancestral/family line can be traced by testing a DNA type of interest;
vii) selecting an individual from said set and determining which ancestral/family line can be traced by testing each DNA type carried by said individual;
viii) selecting a first individual from said set and determining which additional individuals in said set belong to the same ancestral/family line as said first individual;
ix) selecting a first individual from said set and determining which additional individual in said set carries the same DNA characteristic for said DNA type of interest as said first individual;
x) selecting a first individual from said set and determining which other individuals in said set carry DNA which can provide information for said individual;
xi) selecting a first individual from said set and determining which information for which other individual from said set can be derived from testing said first individual;
xii) selecting a first individual from said set, selecting a second individual from said set and determining which DNA type has the same DNA characteristic for said first individual and said second individual;
xiii) selecting a first individual from said set, selecting a second individual from said set, selecting at least one DNA type and determining whether the DNA characteristic for said at least one DNA type is the same for the said first individual and said second individual;
xiv) selecting an individual from said set, selecting a DNA type and determining whether said individual carries said DNA type;
xv) selecting an individual from said set, selecting a DNA type and determining whether said individual can be tested for said DNA type;
xvi) selecting a first individual from said set, selecting a DNA type and determining which additional individual carries the same DNA characteristic for said DNA type as the said first individual;
xvii) selecting an individual from said set, selecting a DNA type and determining which ancestral/family lineage is traced by testing said individual and said DNA type;
xviii) selecting an individual from said set, selecting ancestral/family line for investigation, determining whether said individual carries the DNA type required to trace said ancestral/family line;
xix) selecting a first individual from said set, selecting an ancestral/family line, determining which additional individual is in the same ancestral/family line as the said first individual;
xx) selecting an ancestral/family line of interest and determining which individual in said set carries the DNA type required to trace said ancestral/family line; and
xxi) selecting an ancestral/family line and determining which individual and which DNA type is required to trace said ancestral/family line.

3. The method of claim 1 wherein said set of living or deceased individuals is a family tree.

4. The method of claim 1 wherein said set of living or deceased individuals is a family list.

5. The method of claim 3 wherein at least one of the following information can be viewed from the family tree:
a) the carriers of genetic information;
b) the DNA characteristics for the DNA type for at least one said individual;
c) the initiation process for genetic analysis.

6. The method of claim 3 wherein the lines to be traced and the test status of said at least one DNA type for the line can be viewed in the family tree.

7. The method of claim 1 wherein a plurality of individuals in the set are linked in an interactive computerized network.

8. The method of claim 7 wherein said interactive computerized network is a social network.

9. The method of claim 8 wherein a plurality of users are accessible to the social network.

10. The method of claim 3 wherein one or more than one of said individuals in said family tree are linked in an interactive computerized network.

11. The method of claim 10 wherein one or more than one of said individuals in said family tree are linked in a social network.

12. The method of claim 1 wherein genetic testing can be initiated for one or more than one individual in the set.

13. The method of claim 12 wherein said genetic testing involves carrying out at least one of the following steps:
a) identifying the individual for genetic testing;
b) identifying the DNA type for genetic testing;
c) identifying the availability of the said individual for genetic testing;
d) identifying the ability to sample DNA from the said individual for genetic testing;
e) identifying the ability to obtain a sample containing the DNA from the said individual for genetic testing;
f) ordering a genetic test for the said individual for at least one DNA type;
g) obtaining a genetic test for an individual in the system.

14. The method of claim 12 wherein the genetic testing can be initiated by notifying another individual to initiate genetic testing.

15. The method of claim 12 wherein one or more than one individual in the set can initiate genetic testing on the individual himself or herself.

16. The method of claim 12 wherein one or more than one individual in the set can initiate genetic testing for one or more than one other individuals in the set.

17. The method of claim 10 wherein the initiation of genetic testing is facilitated by the computerized network.

18. The method of claim 12 wherein said genetic testing can be conducted on the DNA of said individual to determine the DNA characteristic of said one or more than one DNA type.

19. The method of claim 12 wherein the initiation of genetic testing can be by means of ordering a genetic test or asking another individual to order a genetic test.

20. The method of claim 12 wherein an individual can access the DNA information for himself or herself.

21. The method of claim 12 wherein one or more than one individual can access the DNA information for one or more than one additional other individual in the set.

22. The method of claim 12 wherein one or more than one individual in the set can use the system to determine who can be tested, and then either ask the individual to test or facilitate the testing for the individual.

23. The method of claim 1 wherein family lines can be defined by the type of genealogical relationships between individuals in the set.

24. The method of claim 3 wherein the carriers of genetic information can be viewed in the family tree.

25. The method of claim 3 wherein a family line to be traced can be viewed in the family tree.

26. The method of claim 1 further comprising the step of sampling the DNA of least one carrier of said DNA type of interest in said set.

27. The method of claim 26 further comprising the step of determining what information the testing of an individual in the set will contribute prior to sampling the DNA of at least one said carrier.

28. The method of claim 27 further comprising the step of determining whether said carrier is living or deceased, and if said carrier is deceased then determining if said deceased carrier's genetic information is known, and if not, then determining whether a forensic DNA sample can be obtained and, if so, obtaining said forensic DNA sample for analysis.

29. The method of claim 1 wherein said DNA type of interest is Y-DNA and a male is specified as the carrier of Y-DNA.

30. The method of claim 29 wherein the paternal line of a first individual in said set is traced by determining whether said first individual carries Y-DNA and whether said first individual can be tested.

31. The method of claim 30 comprising the further step of determining whether any other individuals in said set carries Y-DNA with the same characteristics as the Y-DNA of said first individual and whether said other individuals can be tested.

32. The method of claim 1 wherein said DNA type of interest is mtDNA and a male or female is specified as the carrier of mtDNA.

33. The method of claim 32 wherein the maternal line of a first individual in said set is traced by determining whether said first individual carries mtDNA and whether said first individual can be tested.

34. The method of claim 33 comprising the further step of determining whether any other individuals in said set carries mtDNA with the same characteristics as the mtDNA of said first individual and whether said other individuals can be tested.

35. The method of claim 1 comprising adding further individuals to the set until one or more than one individual who carries the DNA type of interest is identified.

36. The method of claim 35 wherein additional individuals are added to the set of individuals related by defined genealogical relationships until at least one individual who carries a DNA marker required to trace the ancestral/family line of interest is added.

37. The method of claim 1 further comprising the step of determining whether it is possible to sample the DNA from at least one of said individuals for DNA testing.

38. The method of claim 37 wherein when the DNA of said at least one of said individuals cannot be sampled for DNA testing, additional individuals in said set which carry the same DNA type as the said first individual are determined and the possibility of sampling the DNA of said additional individuals is determined.

39. The method of claim 37 wherein when the DNA of said individual cannot be sampled, a forensic specimen containing the DNA of said individual is tested.

40. The method of claim 38 wherein when the DNA of said additional individual cannot be sampled, a forensic specimen containing the DNA of said individual is tested.

41. The method of claim 26 further comprising the step of determining whether said carrier is living or deceased, and if said carrier or additional carrier is deceased, then determining if said deceased carrier's genetic information is known, and if not, then determining whether a forensic DNA sample can be obtained, and if so, obtaining said forensic DNA sample for analysis.

42. The method of claim 1 wherein said inquiry comprises determining the possibility of tracing an ancestral/family line of interest and determining the individual who carries the DNA type required to trace said ancestral/family line of interest.

43. The method of claim 42 wherein when no individual in the set carries the DNA type required to trace the ancestral/family line, individuals are added to the set and the inquiry in claim 40 is repeated until at least one individual who carries the DNA type required to trace the ancestral/family line of interest is identified.

44. The method of claim 42 wherein additional individuals are added to the set of individuals related by defined genealogical relationships until an individual who carries the DNA marker required to trace the ancestral/family line of interest is added.

45. A computer-implemented system for conducting genetic investigation using a set of individuals related by defined genealogical relationships, comprising:
a) computer-implemented means for defining and storing a set of living or deceased individuals related by defined genealogical relationships;
b) computer-implemented means for defining at least one DNA type of interest;
c) computer-implemented means for defining the inheritance pattern associated with each said at least one DNA type of interest;
d) computer-implemented means for defining the information associated with each said at least one DNA type of interest;
e) computer-implemented means for obtaining genetic information in relation to said set of living or deceased individuals, by carrying out a chain of inquiry using said at least one DNA type of interest, and said inheritance pattern associated with each said at least one DNA type of interest.
